(19) United States
(12) Patent Application Publication
(10) Pub. No.: US 2024/0002864 A1
(43) Pub. Date: Jan. 4, 2024

(54) Systems and Methods for Enhancing Gene Expression

(71) Applicant: The Board of Trustees of the Leland Stanford Junior University, Stanford, CA (US)

(72) Inventors: Maria Barna, Stanford, CA (US); Kathrin Leppek, Stanford, CA (US)

(73) Assignee: The Board of Trustees of the Leland Stanford Junior University, Stanford, CA (US)

(21) Appl. No.: 17/998,789

(22) PCT Filed: May 11, 2021
(86) PCT No.: PCT/US2021/031875

§ 371 Date: Nov. 14, 2022

Related U.S. Application Data
(60) Provisional application No. 63/022,898, filed on May 11, 2020.

(51) Int. Cl. C12N 15/67

(52) U.S. Cl. CPC C12N 15/67; C12N 2310/531

(57) ABSTRACT

Systems and methods for enhancing mRNA translation are disclosed. Some embodiments describe expression constructs for producing a peptide and include a translational enhancer. Additional embodiments describe methods for producing a peptide using a construct including a translational enhancer.

### CROSS-REFERENCE TO RELATED APPLICATIONS

The current application claims priority to U.S. Provisional Patent Application No. 63/022,898, entitled “Systems and Methods for Enhancing Gene Expression” to Maria Barna et al., filed May 11, 2020, the disclosure of which is hereby incorporated by reference in its entirety.

### STATEMENT REGARDING FEDERALLY SPONSORED RESEARCH OR DEVELOPMENT

This invention was made with Governmental support under Contract No. HD086634 awarded by the National Institutes of Health. The government has certain rights in the invention.

### FIELD OF THE DISCLOSURE

The present disclosure relates to gene regulation, and in particular methods, systems, and compositions enhance gene regulation.

### INCORPORATION OF SEQUENCE LISTING

A computer readable form of the sequence listing, “06738PCT_Seq_List_ST25.txt”, submitted via EFS-WEB, is herein incorporated by reference in its entirety.

### BACKGROUND OF THE DISCLOSURE

Messenger RNA (mRNA) based therapeutics hold the potential to transform modern medicine because of their fast production and use for precise therapies involving reprogramming patients' own cells to produce therapeutic proteins. Compared to the development of recombinant proteins, production of mRNA is faster, more cost-effective, and more flexible because it can be easily produced by in vitro transcription. However, technical obstacles facing mRNA pharmaceuticals are also apparent. These obstacles include the optimization of the stability, translation efficiency, and delivery mechanisms for RNA therapeutics, which are all pivotal issues that need to be carefully optimized for preclinical and clinical applications. For example, mRNA vaccines still suffer from decreased efficacy due to poor expression of the payload m RNA. Poor expression creates an obstacle to dosing of mRNA-based therapeutics that has not been resolved.

### SUMMARY OF THE DISCLOSURE

This summary is meant to provide examples and is not intended to be limiting of the scope of the invention in any way. For example, any feature included in an example of this summary is not required by the claims, unless the claims explicitly recite the feature. Also, the features described can be combined in a variety of ways. Various features and steps as described elsewhere in this disclosure can be included in the examples summarized here.

Systems and methods for enhancing gene expression are disclosed. In one embodiment, a construct to enhance gene translation includes a coding region and a 5′-UTR located at the 5′ end of the coding region and including at least one translational enhancer.

In a further embodiment, the 5′-UTR further includes a spacer located between the translational enhancer and the coding region.

In another embodiment, the spacer is approximately 100-150 nt in length.

In a still further embodiment, the spacer is selected from the group consisting of: a Hoxa9 native spacer (SEQ ID NO: 15), an actin 5′-UTR (SEQ ID NO: 16), and an inverted actin 5′-UTR (SEQ ID NO: 17).

In still another embodiment, the translational enhancer is a Hoxa9 IRES-like element.

In a yet further embodiment, the translational enhancer is a stem-loop structure isolated from a Hox gene.

In yet another embodiment, the translational enhancer is SEQ ID NO: 1 or a sequence variant thereof.

In a further embodiment again, the translational enhancer is selected from SEQ ID NOs: 2-14.

In another embodiment again, the translational enhancer possesses at least 75% sequence identity to SEQ ID NO: 1.

In a further additional embodiment, a method for producing a peptide includes obtaining an expression construct possessing a target gene and a 5′-UTR, where the expression construct includes a coding region and a 5′-UTR located at the 5′ end of the coding region and including at least one translational enhancer, and delivering the expression construct to a ribosome for translation.

In another additional embodiment, the 5′-UTR further includes a spacer located between the translational enhancer and the coding region.

In a still yet further embodiment, the spacer is approximately 100-150 nt in length.

In still yet another embodiment, the spacer is selected from the group consisting of: a Hoxa9 native spacer (SEQ ID NO: 15), an actin 5′-UTR (SEQ ID NO: 16), and an inverted actin 5′-UTR (SEQ ID NO: 17).

In a still further embodiment again, the translational enhancer is a Hoxa9 IRES-like element.

In still another embodiment again, the translational enhancer is a stem-loop structure isolated from a Hox gene.

In a still further additional embodiment, the translational enhancer is SEQ ID NO: 1 or a sequence variant thereof.

In still another additional embodiment, the translational enhancer is selected from SEQ ID NOs: 2-14.

In a yet further embodiment again, the translational enhancer possesses at least 75% sequence identity to SEQ ID NO: 1.

In yet another embodiment again, the method further includes isolating a peptide produced by the ribosome using the expression cassette.

In a yet further additional embodiment, a medical formulation includes an RNA molecule including a coding region and a 5′-UTR located at the 5′ end of the coding region and including at least one translational enhancer.

In yet another additional embodiment, the medical formulation further includes one or more of a buffer, a lubricant, a binder, a flavorant, and a coating.

In a further additional embodiment again, the formulation is delivered to an individual orally, nasally, inhalationally, parentally, intravenously, intraperitoneally, subcutaneously, intramuscularly, intradermally, topically, rectally, intracerebrally, intraventricularly, intracerebroventricularly, intrathecally, intracisternally, intraspinally, perispinally, intraocularly, or intravitreally.

In another additional embodiment again, the 5′-UTR further includes a spacer located between the translational enhancer and the coding region.

In a still yet further embodiment again, the spacer is approximately 100-150 nt in length.

In still yet another embodiment again, the spacer is selected from the group consisting of: a Hoxa9 native spacer (SEQ ID NO: 15), an actin 5′-UTR (SEQ ID NO: 16), and an inverted actin 5′-UTR (SEQ ID NO: 17).

In a still yet further additional embodiment, the translational enhancer is a Hoxa9 IRES-like element.

In still yet another additional embodiment, the translational enhancer is a stem-loop structure isolated from a Hox gene.

In a yet further additional embodiment again, the translational enhancer is SEQ ID NO: 1 or a sequence variant thereof.

In yet another additional embodiment again, the translational enhancer is selected from SEQ ID NOs: 2-14.

In a still yet further additional embodiment again, the translational enhancer possesses at least 75% sequence identity to SEQ ID NO: 1.

The foregoing and other objects, features, and advantages of the disclosed technology will become more apparent from the following detailed description, which proceeds with reference to the accompanying figures.

### BRIEF DESCRIPTION OF THE DRAWINGS

FIGS. 1A-1K illustrate details regarding translational enhancers in accordance with various embodiments of the invention.

FIGS. 2A-2F illustrate details regarding the ability of translational enhancers to bind to ribosomal RNA (rRNA) in accordance with various embodiments of the invention.

FIG. 3 illustrates an expression construct in accordance with various embodiments of the invention.

FIG. 4 illustrates a method for producing a peptide or protein in accordance with various embodiments of the invention.

### DETAILED DESCRIPTION OF THE DISCLOSURE

Translation involves an interaction between mRNA, which code for certain proteins or peptides, and ribosomes, which assemble a peptide from the mRNA sequence. The mechanisms of eukaryotic translation initiation and principles of its regulation are of great interest both with respect to new layers of control to gene expression as well as for the discovery of novel sequences and structures that can boost the translation of downstream open reading frames. Such translation regulatory regions can be extended to the design of RNA vaccines, viral-based therapies, as well as the production of any protein in cells and organisms. Additionally, mRNAs containing optimized UTRs for increased expression will reduce the burden on rapid mass production of therapeutic mRNAs.

The ribosome is built from proteins and RNA. The latter is transcribed from ribosomal DNA (rDNA) consisting of hundreds of tandemly repeated copies. Importantly, a dramatic change occurred during the course of eukaryotic evolution, which increased the overall size of the ribosome. For example, the human ribosome is over 1 MDa larger than the yeast ribosome. This is due in part to the insertions of blocks of sequences that are called expansion segments (ESs), as they “expand” the eukaryotic rRNA. (See e.g., Gerbi, S. A. (1996). Expansion segments: regions of variable size that interrupt the universal core secondary structure of ribosomal RNA. In Ribosomal RNA: Structure, Evolution, Processing and Function in Protein Synthesis, Eds: R. A. Zimmermann and A. E. Dahlberg. Telford—CRC Press, Boca Raton, FL., pp. 71-87; the disclosure of which is incorporated herein by reference in its entirety.) ESs are located in rRNA regions of low primary sequence conservation, which implies that they are tolerated because they do not interfere with essential rRNA function. Although ESs are generally found within the same location in the rRNAs of different eukaryotes, they can exhibit a striking degree of variability as they vary in their length and sequence both within and among different species, including different tissue types. (See e.g., Kuo, B. A., et al. (1996). Human ribosomal RNA variants from a single individual and their expression in different tissues. Nucleic Acids Res. 24, 4817-4824; Leffers, H., and Andersen, A. H. (1993). The sequence of 28S ribosomal RNA varies within and between human cell lines (Nucleic Acids Res. 21, 1449-1455; and Parks, M. M., et al. (2018). Variant ribosomal RNA alleles are conserved and exhibit tissue-specific expression. Sci. Adv. 4, eaao0665, 1-13; the disclosures of which are incorporated by reference herein in their entireties.) The longest of ESs are more than 700 nt in Homo sapiens (H. sapiens) and resemble tentacle-like, highly flexible extensions (Anger, A. M., et al. (2013). Structures of the human and Drosophila 80S ribosome. Nature 497, 80-85; Armache, J.-P., et al. (2010). Cryo-EM structure and rRNA model of a translating eukaryotic 80S ribosome at 5.5-A resolution. Proc. Natl. Acad. Sci. U.S.A 107, 19748-19753; and Gerbi, S. A. (1996). Expansion segments: regions of variable size that interrupt the universal core secondary structure of ribosomal RNA. In Ribosomal RNA: Structure, Evolution, Processing and Function in Protein Synthesis, Eds: R. A. Zimmermann and A. E. Dahlberg. Telford—CRC Press, Boca Raton, FL., pp. 71-87; the disclosures of which are incorporated by reference herein in their entireties.) Whether ESs have a functional role in translational control as well as the significance of their dramatic variability across evolution remains poorly understood. Indeed, it has been argued that, due to their extensive variation in sequence and length, ESs may reflect neutral mutations at non-essential domains of the ribosome and thereby, for the last several decades, there had been a very limited understanding of a critical facet in the evolution of an ancient molecular machinery and its biological impact on gene regulation and organismal development. (See e.g., Gerbi, S. A. (1986). The evolution of eukaryotic ribosomal DNA. BioSystems 19, 247-258; the disclosure of which is incorporated herein by reference in its entirety.)

As master regulators of metazoan body plan formation, Hox gene clusters of transcription factors are one of the most spatially and temporally regulated transcripts. (See e.g., Mallo, M., and Alonso, C. R. (2013). The regulation of Hox gene expression during animal development. Development 140, 3951-3963; the disclosure of which is incorporated herein by reference in its entirety.) A subset of Hox transcripts within the Hoxa cluster, contain structured RNA internal ribosome entry sites (IRES)-like elements. (See e.g., Xue, S., et al. (2015). RNA regulons in Hox 5′ UTRs confer ribosome specificity to gene regulation. Nature 517, 33-38; the disclosure of which is incorporated herein by reference in its entirety.) These regulatory elements recruit the ribosome and direct cap-independent translation critical for Hox gene expression and early anterior-posterior patterning of the axial skeleton. (See e.g., Leppek, K., et al. (2017). Functional 5′-UTR mRNA structures in eukaryotic translation regulation and how to find them. Nat. Rev. Mol. Cell Biol. 19, 158-174; and Plank, T. D. M., and Kieft, J. S. (2012). The structures of nonprotein-coding RNAs that drive internal ribosome entry site function. Wiley Interdiscip. Rev. RNA 3, 195-212; Kondrashov, N., et al. (2011). Ribosome-mediated specificity in Hox mRNA translation and vertebrate tissue patterning. Cell 145, 383-397; and Xue, S., et al. (2015). RNA regulons in Hox 5′ UTRs confer ribosome specificity to gene regulation. Nature 517, 33-38; the disclosures of which are incorporated by reference herein in their entireties.) The presence of a cap-proximal Translation Inhibitory Element (TIE), a potent repressor of cap-dependent translation, within these 5′-UTRs, allows these Hox genes to be translated by the downstream IRES-like element as a means to more specifically control their spatiotemporal translation in development.

Turning now to the drawings and data, embodiments herein are directed to methods, systems, and compositions to enhance gene regulation. Many embodiments utilize interactions between ribosomes (rRNA) and messenger RNA (mRNA) to increase translation of the mRNA. In certain embodiments, the interactions between ribosomes are between ribosomal RNA (rRNA) and mRNA. Additional embodiments utilize expansion segments (ESs) located in the rRNA as the basis for the interactions. In various embodiments, the mRNA contains a particular sequence in the 5′-untranslated region (5′-UTR) of the mRNA that interacts with an ES in the rRNA. In many embodiments the sequence in one or more of the ES and 5′-UTR form a secondary structure that interact. In certain embodiments, the particular sequence in the 5′-UTR is specific to a particular species, including humans. Further embodiments include methods to design, test, and/or confirm an interaction between ribosomes and mRNAs.

### 5′-UTR Sequences for Translational Enhancement

Many embodiments are directed to sequences located in a 5′-UTR to enhance RNA translation in a cap-independent manner and/or a cap-dependent manner. Certain embodiments utilize one or more IRES-like elements to increase the translation. Some of these embodiments form stem-loop structures that interact with rRNA regions (including ESs). Certain embodiments use one or more pairing (P) elements from genes showing RES activity, including Homeobox (Hox) genes, such as Hoxa9. Certain stress states, including hypoxia, nutrient deprivation, viral infections, during radiation therapy or chemotherapy, and other stresses can cause a deregulation of cap-dependent translation. As such, cap-independent translation of certain embodiments overcomes these challenges in gene expression and mRNA translation. Additionally, increasing cap-dependent translation in some embodiments is effective for the design of RNA vaccines, RNA-based therapies, and exogenous expression of proteins directed by these regulatory elements, including but limited to any plasmid or expression cassette.

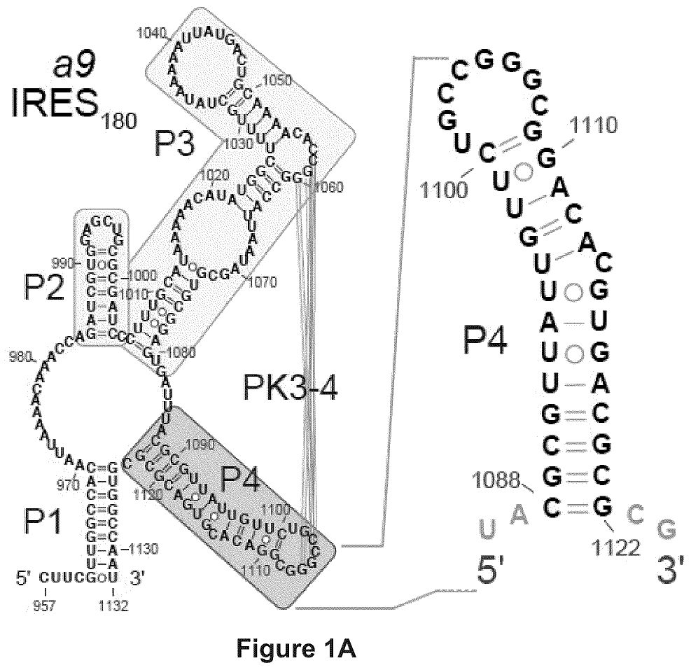

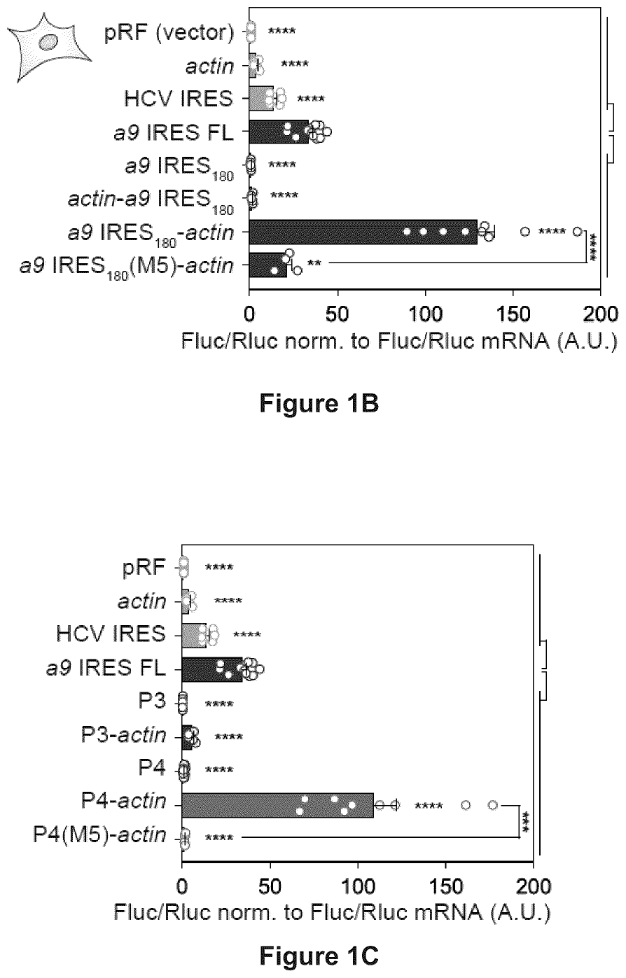

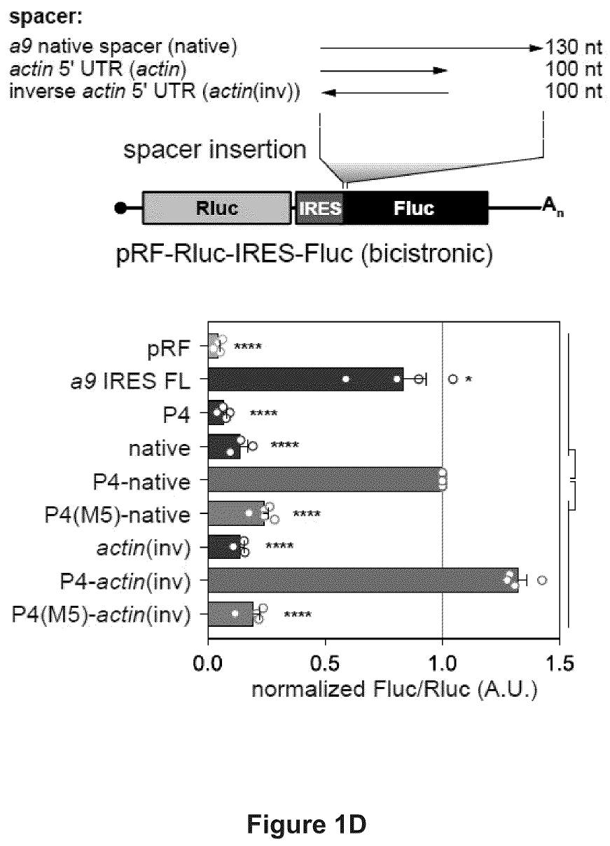

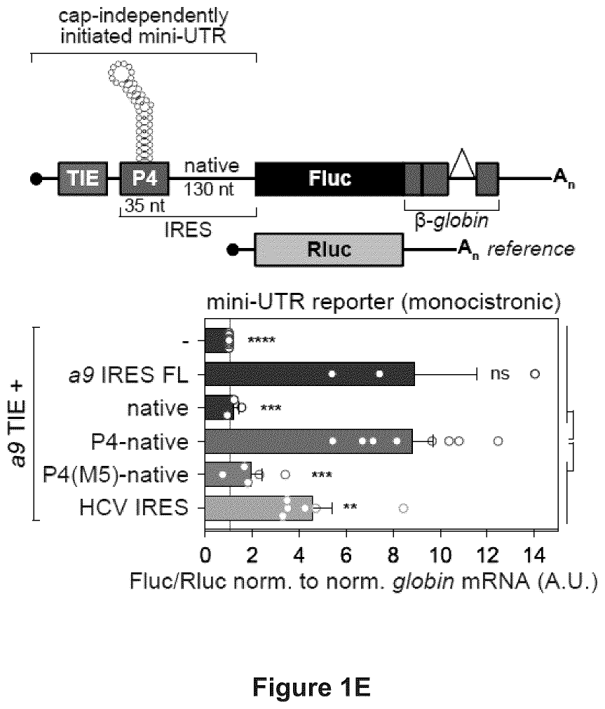

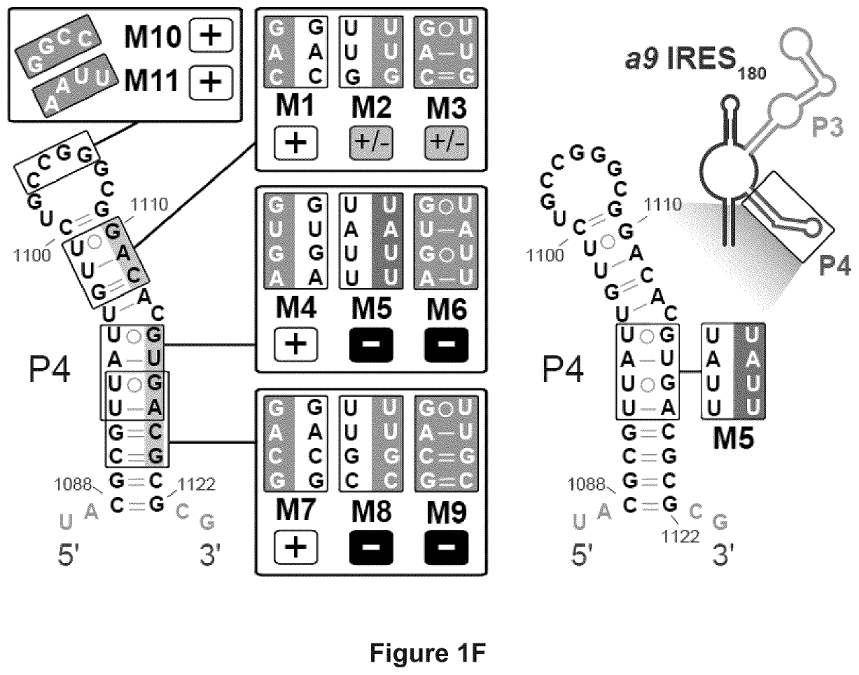

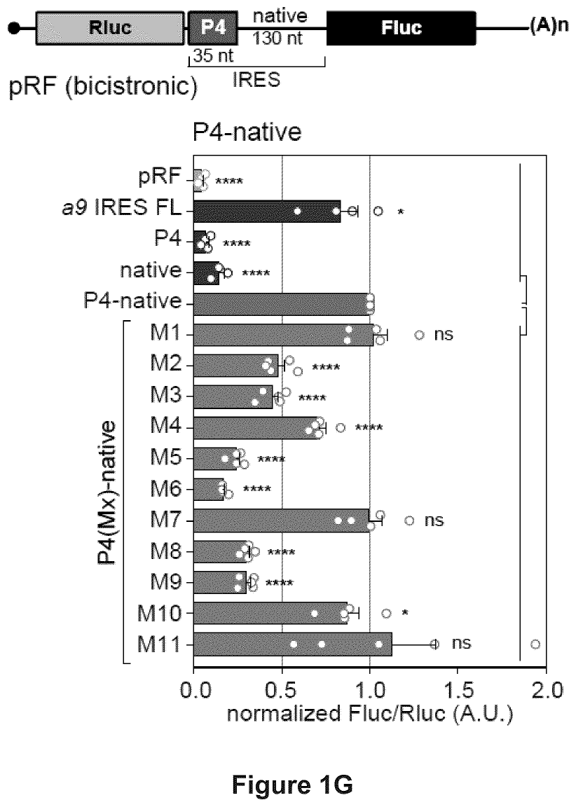

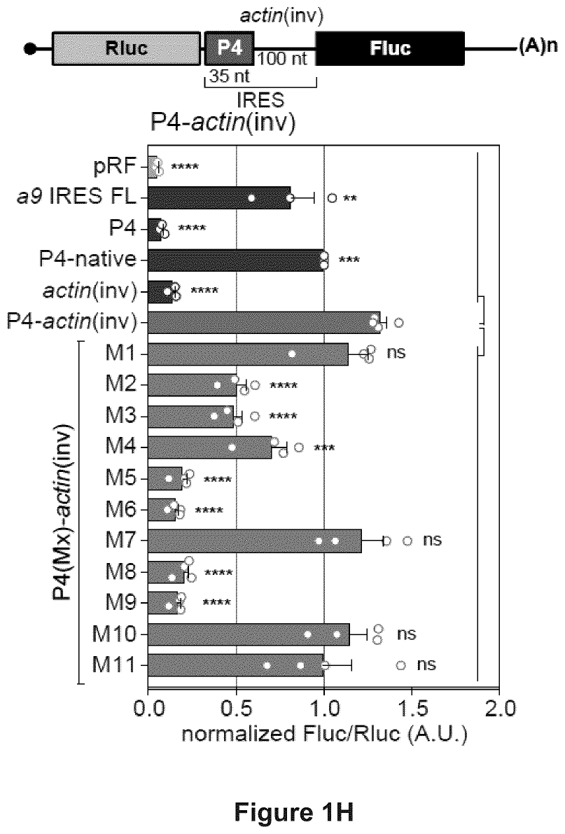

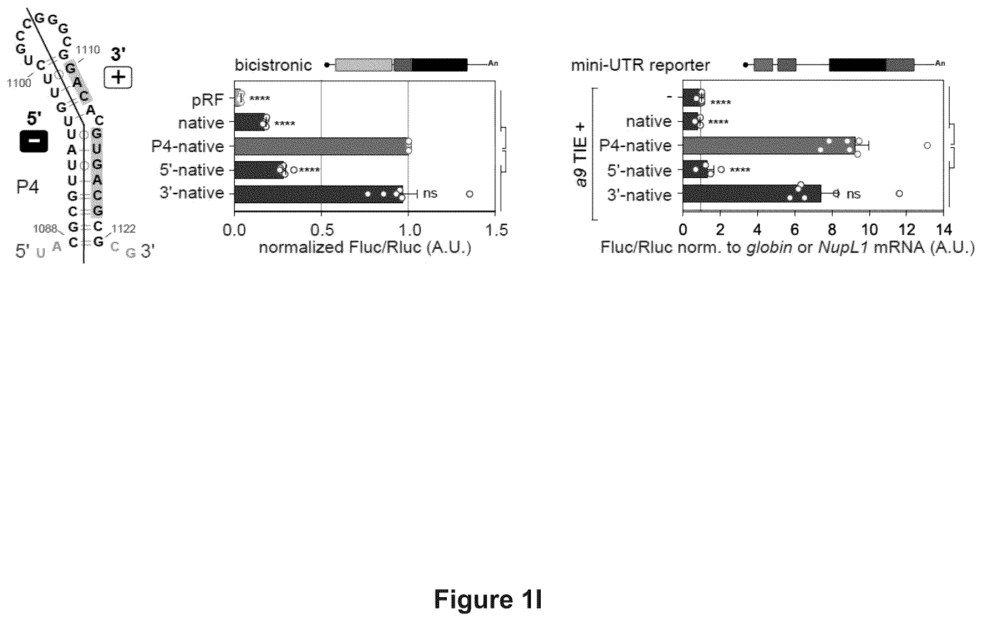

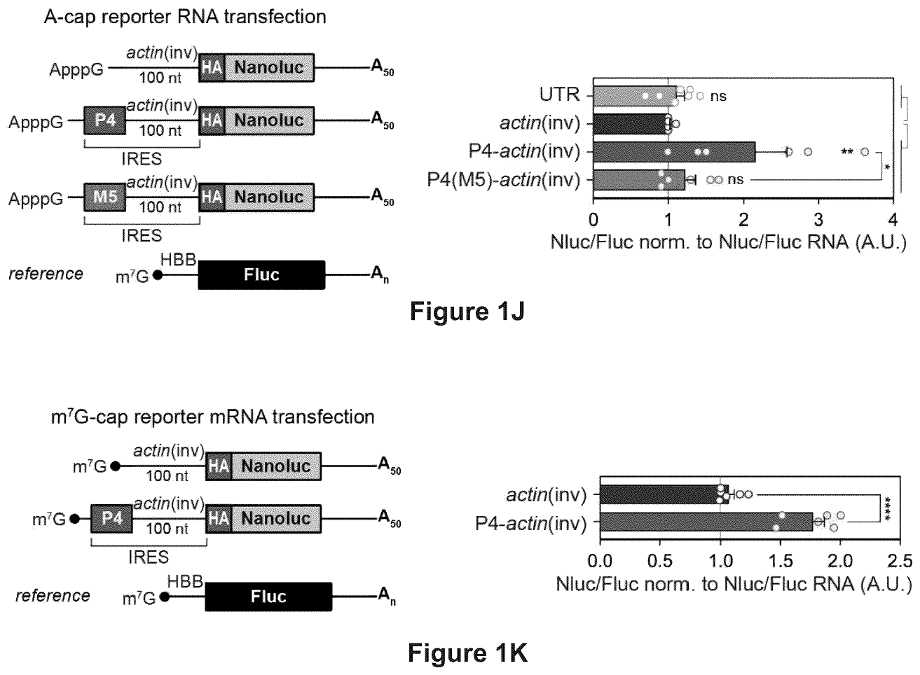

Turning to FIG. 1A, the highly conserved mouse Hoxa9 IRES-like element folds into a 180 nucleotide (nt) long RNA secondary structure (a9 IRES₁₈₀), that includes four pairing (P) elements P1-P4. (See e.g., Cheng, C. Y., et al. (2015). Consistent global structures of complex RNA states through multidimensional chemical mapping. Elife 4, 1-38; andXue, S., et al. (2015). RNA regulons in Hox 5′ UTRs confer ribosome specificity to gene regulation. Nature 517, 33-38; the disclosures of which are incorporated by reference herein in their entireties.) In accordance with many embodiments, the Hox9a IRES-like element is used as the basis of a 5′-UTR for enhanced mRNA translation. However, while the a9 IRES₁₈₀ element is sufficient for IRES-like activity, it is dependent on the distance from the start codon, as illustrated in FIG. 1B, where a 100 nt 5′-UTR from actin rescued m RNA translation. Additionally, the P4 element (SEQ ID NO: 1), which is a 35 nt stem-loop, including an actin spacer (SEQ ID NO: 16) is also sufficient for IRES-like activity, such as illustrated in FIG. 1C. Turning to FIG. 1D, full length (including a native 130 nt spacer (SEQ ID NO: 15)) a9 IRES (a9 IRES FL), native P4 (including the native 130 nt spacer (SEQ ID NO: 15)), and a P4 with an inverted actin 5′-UTR spacer (SEQ ID NO: 17) exhibit increased mRNA translation, in accordance with many embodiments.

Turning to FIG. 1E, mRNA translation of constructs including a reporter mRNA are illustrated. These constructs include a TIE at the 5′ end to suppress cap-dependent translation. Under such a construction, full length a9 IRES and P4 with a native, 130 nt, spacer (SEQ ID NO: 15) both show increased levels of translation, indicating that some embodiments are capable of cap-independent translation.

Additional embodiments use mutated forms of one or more pairing elements to alter mRNA translation. For example, FIG. 1F illustrates several sequence variations within P4 in accordance with certain embodiments that are active (+), inactive (−), or moderately active (+/−) translation levels of the mRNA relative to a native P4 element, as shown in FIGS. 1G and 1H. Modified sequences M1-M11 in accordance with various embodiments are included as SEQ ID NOs: 2-12.

Further embodiments include just one arm of the P4 stem-loop to alter mRNA translation. FIG. 11 illustrates a native P4 element bisected to indicate the 3′-arm (SEQ ID NO: 13) and 5′-arm (SEQ ID NO: 14) in accordance with some embodiments. Further, translation is increased when just the native 3′-arm to comparable levels as the entire P4 element, whether in a bicistronic or with a mini-UTR reporter mRNA.

Turning to FIG. 1J-1K, cap-dependent and cap-independent translation of certain embodiments are illustrated. In particular, FIG. 1J illustrates cap-independent translation enhancement of many embodiments, where a 5′-UTR including P4 (SEQ ID NO: 5) concatenated to an inverse actin 5′-UTR (SEQ ID NO: 17) increases translation when in a construct with an “A-Cap” (ApppG) cap, which prohibits cap-dependent translation. (See e.g., Hundsdoerfer, P., Thoma, C., and Hentze, M. W. (2005). Eukaryotic translation initiation factor 4GI and p97 promote cellular internal ribosome entry sequence-driven translation. Proc. Natl. Acad. Sci. U.S.A 102, 13421-134; the disclosure of which is incorporated by reference herein in its entirety.) FIG. 1K illustrates translational enhancement in a cap-dependent manner, where a 5′-UTR including P4 (SEQ ID NO: 5) concatenated to an inverse actin 5′-UTR (SEQ ID NO: 17) increases translation when in a construct with a canonical, m⁷G, cap on the mRNA. Because certain embodiments exhibit translational enhancement in both A-capped and canonically capped mRNAs, many embodiments are capable of enhancing translation in cap-dependent and cap-independent manners.

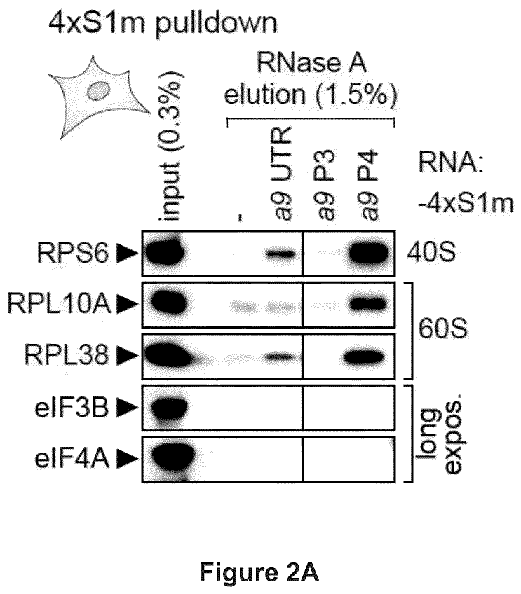

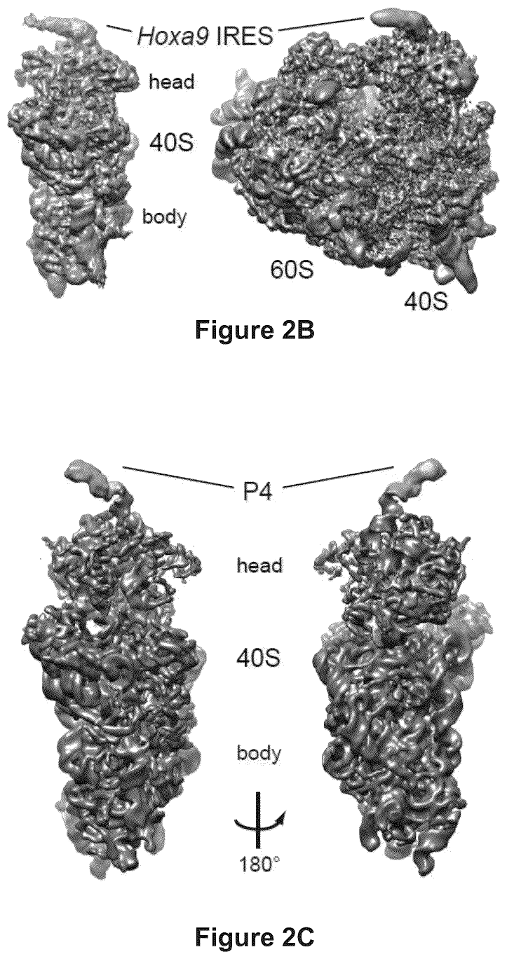

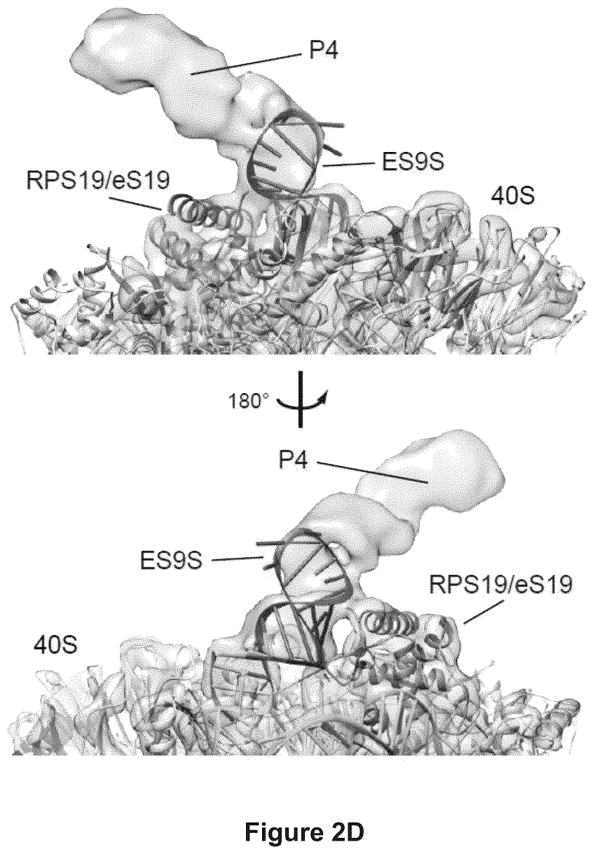

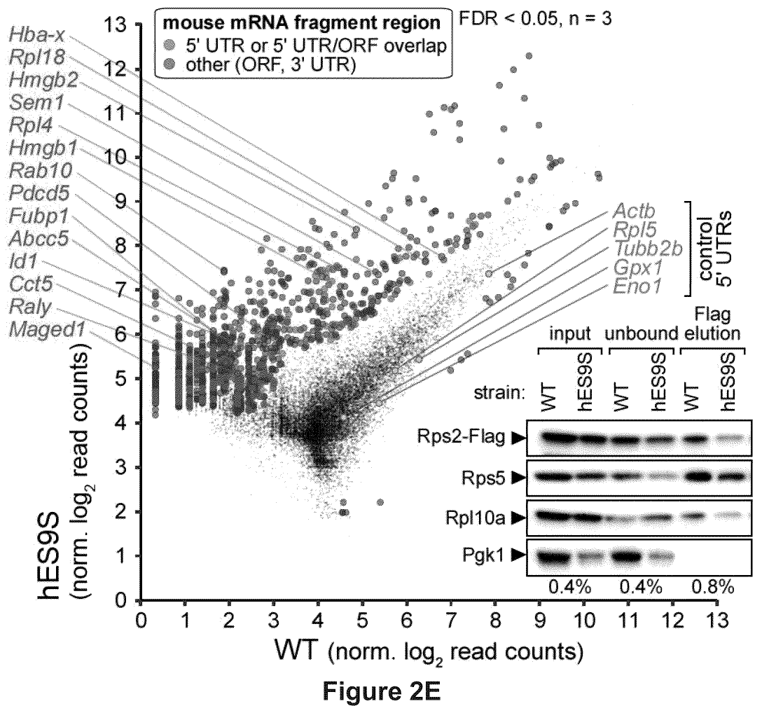

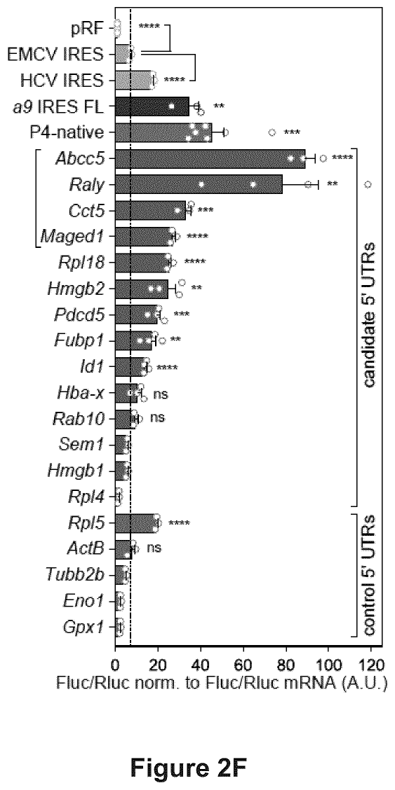

Turning to FIGS. 2A-2D, many embodiments of 5′-UTR translation enhancers interact with the ribosome. In particular, FIG. 2A illustrates a western blot showing interaction of an a9 UTR as well as P4 element (SEQ ID NO: 1) with the 40S and 60S ribosomal subunits in accordance with certain embodiments. Additionally, FIGS. 2B-2C illustrate cryo-EM reconstructions of interactions between ribosomes and a full length a9 IRES (FIG. 2B) and P4 (FIG. 2C). Similarly, FIG. 2D illustrates a closer examination of the interaction between some embodiments with a particular ES (ES9S), indicating that many embodiments of translation enhancers interact with rRNA.

Additional embodiments are selected from 5′-UTRs that show an interaction with ES9S. FIG. 2E illustrates RNA fragments in accordance with many embodiments from 460 genes that show an affinity for ES9S and differentiated based on a location in a or other segment of the mRNA. Certain embodiments selected for genes where the sequence showing ES9S affinity was present in the 5′-UTR, which are listed in Table 1, and FIG. 2F illustrates IRES activity of some embodiments where the gene's showed ES9S affinity.

### Constructs for Enhancing Translation

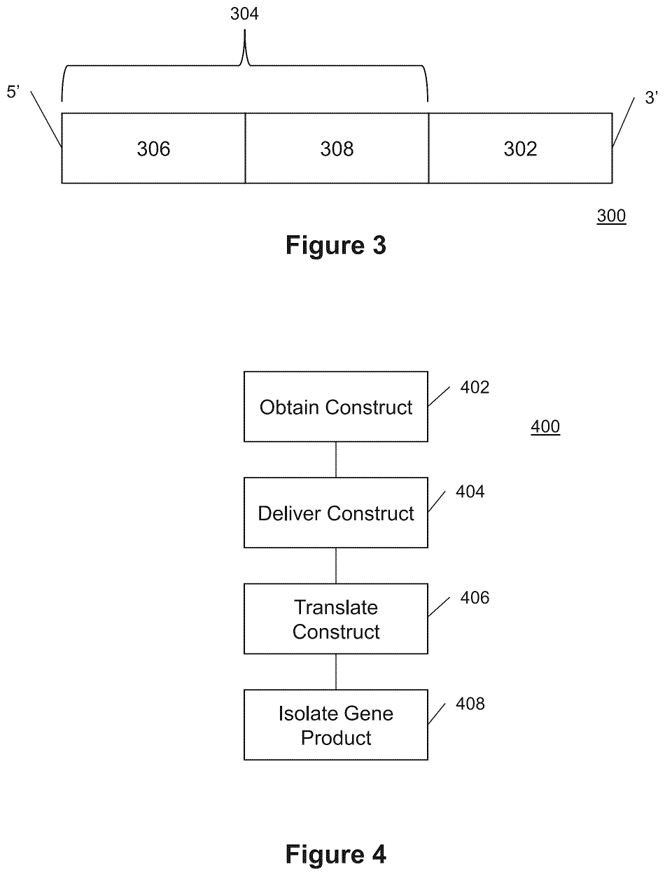

Turning FIG. 3, Many embodiments are directed to expression constructs 300 incorporating translational enhancers. Constructs of numerous embodiments include a coding region 302 and a 5′-UTR 304 located at the 5′ end of coding region 302. In certain embodiments, the coding region 302 is selected for increased production of its resultant protein or peptide and can include a particular gene. In some embodiments, a gene is a natural gene isolated from an organism or species, while certain embodiments the gene is an artificial or designed gene to generate a specific peptide. In many embodiments a 304 includes a translational enhancer 306. In many embodiments, the translational enhancer 306 is an IRES or IRES-like element. In certain embodiments, the translational enhancers 306 possess a stem-loop structure. Numerous embodiments possess a Hoxa9 IRES as the translational enhancer 306, while some embodiments possess a smaller structure. Various embodiments use P4 (SEQ ID NO: 1) as the translational enhancer 306. Certain embodiments use a sequence variant of P4, including, but not limited to SEQ ID NOs: 2-12 as the translational enhancer 306. Some embodiments possess a sequence variant of P4 having at least 75%, 77%, 80%, 85%, 90%, 95%, or 99% sequence identity to P4 (SEQ ID NO: 1) as the translational enhancer 306. Further embodiments have a translational enhancer 306 representing a truncation or arm of a stem-loop structure, such as P4 (SEQ ID NO: 1). In some of these embodiments, the arm is selected from the 3′-arm (SEQ ID NO: 13) or the 5′-arm (SEQ ID NO: 14) of P4 (SEQ ID NO: 1).

In many embodiments, the 5′-UTR 304 further comprises a spacer 308 located between coding region 302 and translational enhancer 306. In many embodiments, spacer 308 is approximately 100-150 nt in length. Certain embodiments use Hoxa9 native spacer (SEQ ID NO: 15), while some embodiments use an actin 5′-UTR in either its native (SEQ ID NO: 16) or inverted (SEQ ID NO: 17) orientation. In many embodiments, an expression construct 300 is made of RNA, such that the construct is translated into a protein or peptide. In some embodiments, an expression construct 300 is made of DNA along with at least one of a promoter, an enhancer, transcription start site, and/or any other components to transcribe DNA to RNA. Additional embodiments include one or more additional features, such as a 5′ cap, a spacer region, 3′ tail, and/or any other features that assist with translation. It should be noted that while certain sequences within SEQ ID NOs: 1-17 are listed as either DNA or RNA, one of skill in the art would understand how to create an RNA construct from a DNA sequence and/or a DNA construct from an RNA sequence, depending on specific need or use for a specific purpose.

### Methods of Producing a Protein or Peptide

FIG. 4 illustrates a method 400 for producing a protein or peptide. Many embodiments obtain an expression construct at 402. Expression constructs are described elsewhere herein and can be DNA, where the construct is transcribed to mRNA for translation, while some embodiments obtain the construct as RNA, which can be imminently translated.

In various embodiments, an expression construct is delivered to a ribosome for translation 404. In some embodiments, the expression construct is delivered to a cell for translation within the cell (e.g., transfection), such as for production of a peptide and/or protein. Certain embodiments mix the construct to a solution including ribosomes, such as cellular lysate, for in vitro expression. Further embodiments deliver the construct to a mammal or other organism for treatment, including (but not limited to) for purposes of introducing viral-based therapies, (e.g., RNA vaccines) or production of a protein or peptide (e.g., gene therapy to replace or supplement innate proteins and/or peptides). In certain embodiments, the construct is encapsulated in a larger structure for delivery and/or incorporation into a cell, such as a capsid, lipid nanoparticle, micelle, bacterium, extracellular vesicle, and/or any other means for delivering the construct. In certain embodiments, delivery is accomplished via microinjection, particle bombardment, or other direct means. In certain embodiments involving the treatment of an individual, an RNA construct can be formulated for a medical use, including by combining it with one or more buffers, lubricants, binders, flavorants, and coatings. Various embodiments an expression construct for specific transfection, such as through a virus (e.g., adeno-associated viruses (AAVs)), viroids, capsids, micelles, and/or larger DNA and/or RNA structures suitable for targeting and/or stability. Various embodiments delivery medical formulations to an individual via one or more paths selected from orally, nasally, inhalationally, parentally, intravenously, intraperitoneally, subcutaneously, intramuscularly, intradermally, topically, rectally, intracerebrally, intraventricularly, intracerebroventricularly, intrathecally, intracisternally, intraspinally, perispinally, intraocularly, intravitreally, and/or any other means to deliver an expression construct most effectively to a tissue, cell, and/or organ being treated.

In some embodiments, the construct is translated 406 to produce a protein or peptide. In various embodiments, translation is accomplished by incubating a culture or reaction tube at an appropriate temperature. In additional embodiments, including where a construct is delivered to an organism, the reaction is allowed to proceed with little monitoring or incubation.

At 408, many embodiments isolate a gene product (e.g., protein or peptide) of the construct. Certain embodiments isolate the gene product by various means, including chromatographic methods, such as size-exclusion and/or ion-exchange chromatography, pulldown methods, and/or other means of isolating a protein from solution.

### Kits for Gene Expression

Certain embodiments are directed to kits to increase gene expression and/or mRNA translation in an organism. Such embodiments include at least one nucleic acid (either RNA or DNA) with a 5′-UTR sequence (e.g., 5′-UTR 304, FIG. 3). In some embodiments, the 5′-UTR is joined to a target gene sequence (e.g., target gene 302, FIG. 3) via ligation, PCR, and/or a combination thereof. In ligation, certain embodiments include an adapter sequence located at the 3′ end of the 5′-UTR, to allow for a complementary sequence to anneal to the adapter sequence. Other embodiments utilize blunt end ligation, especially in single stranded molecules (e.g., RNA), where complementary sequences are not possible. Embodiments employing ligation further include one or more enzymes (e.g., ligases, topoisomerases, etc.) to ligate the ends of the 5′-UTR and the target gene. Further embodiments alter one or more end of the 5′-UTR and/or the target gene to prevent aberrant ligation between a 5′-UTR and target gene. In some of these embodiments, the 5′-UTR includes a blocking modification on the 5′ end of the 5′-UTR to prevent ligation on the 5′ end. Additional embodiments include enzymes and other reagents to modify the target gene by removing and/or adding phosphate groups and hydroxy groups to the 5′ and/or 3′ ends of the target gene to increase appropriate ligation.

In embodiments employing PCR to add a 5′-UTR to a target gene, the 5′-UTR includes a primer sequence for amplification of a target sequence. The primer sequence can be gene-specific primer. Further embodiments employ a universal primer, such that the primer sequence amplifies the target gene regardless of the target gene sequence. In certain embodiments, a universal primer is concatenated to a gene-specific primer sequence. In such embodiments, two PCR reactions can be employed where the first PCR reaction adds the universal primer to the target gene sequence, while the second PCR adds the 5′-UTR onto the universal primer. PCR-based embodiments include enzymes and reagents for a PCR reaction, including NTPs, dNTPs, buffer, and one or more polymerases, as necessary for amplification of a nucleic acid sequences.

Embodiments employing both PCR and ligation may ligate a universal primer on to target gene sequences, followed by amplification to add the 5′-UTR to the target gene sequence.

Further embodiments include components for transfection or introduction of an expression construct (e.g., 5′-UTR-gene construct). Some embodiments include a plasmid or other larger construct for preservation, replication, and/or transfection of the expression construct. Further embodiments include a delivery mechanism for delivering the construct to a cell or organism. Delivery mechanisms in accordance with various embodiments include bacterial vectors, viral vectors, particle bombardment, other means for introducing the expression construct, and combinations thereof.

### DOCTRINE OF EQUIVALENTS

Having described several embodiments, it will be recognized by those skilled in the art that various modifications, alternative constructions, and equivalents may be used without departing from the spirit of the invention. Additionally, a number of well-known processes and elements have not been described in order to avoid unnecessarily obscuring the present invention. Accordingly, the above description should not be taken as limiting the scope of the invention.

Those skilled in the art will appreciate that the foregoing examples and descriptions of various preferred embodiments of the present invention are merely illustrative of the invention as a whole, and that variations in the components or steps of the present invention may be made within the spirit and scope of the invention. Accordingly, the present invention is not limited to the specific embodiments described herein, but, rather, is defined by the scope of the appended claims.

TABLE 1
ENSEMBLE Gene Codes with ES9S affinity in 5′-UTR

| | |
|---|---|
| ENSMUSG00000018160 | ENSMUSG00000028081 |
| ENSMUSG00000025151 | ENSMUSG00000027012 |
| ENSMUSG00000030447 | ENSMUSG00000064871, |
| | ENSMUSG00000062328 |
| ENSMUSG00000064563, | ENSMUSG00000010406 |
| ENSMUSG00000057841 | |
| ENSMUSG00000054717 | ENSMUSG00000028788 |
| ENSMUSG00000022885 | ENSMUSG00000028609 |
| ENSMUSG00000022234 | ENSMUSG00000031633 |
| ENSMUSG00000066551 | ENSMUSG00000021963, |
| | ENSMUSG00000092784 |
| ENSMUSG00000042745 | ENSMUSG00000032594 |
| ENSMUSG00000039473 | ENSMUSG00000021957 |
| ENSMUSG00000073563 | ENSMUSG00000021111 |
| ENSMUSG00000026147 | ENSMUSG00000005732 |
| ENSMUSG00000031985, | ENSMUSG00000069662 |
| ENSMUSG00000090118 | |
| ENSMUSG00000078578 | ENSMUSG00000002102 |
| ENSMUSG00000042541 | ENSMUSG00000032423 |
| ENSMUSG00000028034 | ENSMUSG00000060126 |
| ENSMUSG00000022893 | ENSMUSG00000060860 |
| ENSMUSG00000027593 | ENSMUSG00000048583 |
| ENSMUSG00000019843 | ENSMUSG00000047139 |
| ENSMUSG00000032014 | ENSMUSG00000045128 |
| ENSMUSG00000046229 | ENSMUSG00000039001, |
| | ENSMUSG00000092827 |
| ENSMUSG00000019432, | ENSMUSG00000037916 |
| ENSMUSG00000098499 | |
| ENSMUSG00000061559 | ENSMUSG00000026234 |
| ENSMUSG00000029146 | ENSMUSG00000028692 |
| ENSMUSG00000032305 | ENSMUSG00000024283 |
| ENSMUSG00000029163 | ENSMUSG00000039001 |
| ENSMUSG00000051768 | ENSMUSG00000028426 |
| ENSMUSG00000020307 | ENSMUSG00000030122 |
| ENSMUSG00000069539 | ENSMUSG00000036478 |
| ENSMUSG00000002393 | ENSMUSG00000004071 |
| ENSMUSG00000003269 | ENSMUSG00000018537 |
| ENSMUSG00000070283 | ENSMUSG00000020265, |
| | ENSMUSG00000112241 |
| ENSMUSG00000071646 | ENSMUSG00000033938 |
| ENSMUSG00000022433 | ENSMUSG00000020738 |
| ENSMUSG00000022106 | ENSMUSG00000022858 |
| ENSMUSG00000026134 | ENSMUSG00000028495 |
| ENSMUSG00000026353 | ENSMUSG00000020978 |
| ENSMUSG00000016559 | ENSMUSG00000002778 |
| ENSMUSG00000032437 | ENSMUSG00000024208 |
| ENSMUSG00000028906 | ENSMUSG00000027637 |
| ENSMUSG00000037601, | ENSMUSG00000037563 |
| ENSMUSG00000091228 | |
| ENSMUSG00000063870, | ENSMUSG00000041773 |
| ENSMUSG00000098762 | |
| ENSMUSG00000031708 | ENSMUSG00000044894 |
| ENSMUSG00000018559 | ENSMUSG00000055839 |
| ENSMUSG00000022476 | ENSMUSG00000020029 |
| ENSMUSG00000053931 | ENSMUSG00000032977 |
| ENSMUSG00000020358 | ENSMUSG00000046330 |
| ENSMUSG00000030677 | ENSMUSG00000005732, |
| | ENSMUSG00000116925 |
| ENSMUSG00000030417 | ENSMUSG00000029632 |
| ENSMUSG00000002984 | ENSMUSG00000027671 |
| ENSMUSG00000021379 | ENSMUSG00000038690 |
| ENSMUSG00000027593, | ENSMUSG00000020250 |
| ENSMUSG00000027596 | |
| ENSMUSG00000073702 | ENSMUSG00000026087 |
| ENSMUSG00000096472 | ENSMUSG00000021701 |
| ENSMUSG00000055302 | ENSMUSG00000060601 |
| ENSMUSG00000028851 | ENSMUSG00000031217 |
| ENSMUSG00000006728 | ENSMUSG00000025016 |
| ENSMUSG00000010045 | ENSMUSG00000058558 |
| ENSMUSG00000003068 | ENSMUSG00000048271 |
| ENSMUSG00000029128 | ENSMUSG00000021917 |
| ENSMUSG00000004263, | ENSMUSG00000028333 |
| ENSMUSG00000107478 | |
| ENSMUSG00000046691 | ENSMUSG00000037266 |
| ENSMUSG00000040383 | ENSMUSG00000044533 |
| ENSMUSG00000027248 | ENSMUSG00000071076 |
| ENSMUSG00000040204 | ENSMUSG00000018770 |
| ENSMUSG00000045515 | ENSMUSG00000022403 |
| ENSMUSG00000022205 | ENSMUSG00000005103 |
| ENSMUSG00000029767 | ENSMUSG00000032002 |
| ENSMUSG00000050953 | ENSMUSG00000071180 |
| ENSMUSG00000021282 | ENSMUSG00000063576 |
| ENSMUSG00000022427 | ENSMUSG00000029426 |
| ENSMUSG00000017778 | ENSMUSG00000032042 |
| ENSMUSG00000026047 | ENSMUSG00000028643 |
| ENSMUSG00000002329 | ENSMUSG00000016427 |
| ENSMUSG00000046432 | ENSMUSG00000020921 |
| ENSMUSG00000038976 | ENSMUSG00000032902 |
| ENSMUSG00000031734 | ENSMUSG00000069274 |
| ENSMUSG00000052187 | ENSMUSG00000034892 |
| ENSMUSG00000021578 | ENSMUSG00000031818 |
| ENSMUSG00000029580 | ENSMUSG00000030811 |
| ENSMUSG00000045136 | ENSMUSG00000028953 |
| ENSMUSG00000030120 | ENSMUSG00000038467 |
| ENSMUSG00000018554 | ENSMUSG00000090247, |
| | ENSMUSG00000025350 |
| ENSMUSG00000023307 | ENSMUSG00000049091 |
| ENSMUSG00000038900 | ENSMUSG00000033545 |
| ENSMUSG00000038374 | ENSMUSG00000022698 |
| ENSMUSG00000020069 | ENSMUSG00000063856 |
| ENSMUSG00000055653 | ENSMUSG00000042747 |
| ENSMUSG00000074129 | ENSMUSG00000046668 |
| ENSMUSG00000026434 | ENSMUSG00000020857 |
| ENSMUSG00000060938 | ENSMUSG00000015120 |
| ENSMUSG00000007415 | ENSMUSG00000063787 |
| ENSMUSG00000093904 | ENSMUSG00000022956, |
| | ENSMUSG00000116933 |
| ENSMUSG00000031167 | ENSMUSG00000048540 |
| ENSMUSG00000091537 | ENSMUSG00000063406 |
| ENSMUSG00000024400 | ENSMUSG00000004096 |
| ENSMUSG00000022178 | ENSMUSG00000049751, |
| | ENSMUSG00000113149 |
| ENSMUSG00000026193 | ENSMUSG00000036733, |
| | ENSMUSG00000094365 |
| ENSMUSG00000031715 | ENSMUSG00000029625 |
| ENSMUSG00000025135 | ENSMUSG00000004885 |
| ENSMUSG00000039323 | ENSMUSG00000041688 |
| ENSMUSG00000009741 | ENSMUSG00000006589 |
| ENSMUSG00000031807 | ENSMUSG00000042043 |
| ENSMUSG00000005698 | ENSMUSG00000066568 |
| ENSMUSG00000047945 | ENSMUSG00000026121 |
| ENSMUSG00000003346 | ENSMUSG00000002320 |
| ENSMUSG00000029710 | ENSMUSG00000028568 |
| ENSMUSG00000027852 | ENSMUSG00000026260 |
| ENSMUSG00000024902 | ENSMUSG00000036972 |
| ENSMUSG00000019505 | ENSMUSG00000072772 |
| ENSMUSG00000059248 | ENSMUSG00000090137 |
| ENSMUSG00000021486 | ENSMUSG00000024750 |
| ENSMUSG00000001100 | ENSMUSG00000025289 |
| ENSMUSG00000063524 | ENSMUSG00000105211, |
| | ENSMUSG00000026043 |
| ENSMUSG00000025508 | ENSMUSG00000029922 |
| ENSMUSG00000078941, | ENSMUSG00000074476 |
| ENSMUSG00000052293 | |
| ENSMUSG00000030551 | ENSMUSG00000042570 |
| ENSMUSG00000096210 | ENSMUSG00000029463 |

ENSMUSG00000026150

1. A construct to enhance gene translation, comprising:
a coding region; and
a 5′-UTR located at the 5′ end of the coding region and comprising at least one translational enhancer.

2. The construct of claim 1, wherein the 5′-UTR further comprises a spacer located between the translational enhancer and the coding region.

3. The construct of claim 2, wherein the spacer is approximately 100-150 nt in length.

4. The construct of claim 2, wherein the spacer is selected from the group consisting of: a Hoxa9 native spacer (SEQ ID NO: 15), an actin 5′-UTR (SEQ ID NO: 16), and an inverted actin 5′-UTR (SEQ ID NO: 17).

5. The construct of claim 1, wherein the translational enhancer is one or more of the following: a Hoxa9 IRES-like element, a stem-loop structure isolated from a Hox gene, SEQ ID NO: 1 or a sequence variant thereof, and SEQ ID NOs: 2-14.

6.-8. (canceled)

9. The construct of claim 1, wherein the translational enhancer possesses at least 75% sequence identity to SEQ ID NO: 1.

10. A method for producing a peptide, comprising:
obtaining an expression construct possessing a target gene and a 5′-UTR, wherein the expression construct comprises a coding region and a 5′-UTR located at the 5′ end of the coding region and comprising at least one translational enhancer; and
delivering the expression construct to a ribosome for translation.

11. The method of claim 10, wherein the 5′-UTR further comprises a spacer located between the translational enhancer and the coding region.

12. (canceled)

13. The method of claim 11, wherein the spacer is selected from the group consisting of: a Hoxa9 native spacer (SEQ ID NO: 15), an actin 5′-UTR (SEQ ID NO: 16), and an inverted actin 5′-UTR (SEQ ID NO: 17).

14. The method of claim 10, wherein the translational enhancer is one or more of the following: a Hoxa9 IRES-like element, a stem-loop structure isolated from a Hox gene, SEQ ID NO: 1 or a sequence variant thereof, and SEQ ID NOs: 2-14.

15.-17. (canceled)

18. The method of claim 10, wherein the translational enhancer possesses at least 75% sequence identity to SEQ ID NO: 1.

19. The method of claim 10, further comprising isolating a peptide produced by the ribosome using the expression cassette.

20. A medical formulation comprising:
an RNA molecule comprising:
a coding region; and
a 5′-UTR located at the 5′ end of the coding region and comprising at least one translational enhancer.

21. The medical formulation of claim 20, further comprising one or more of a buffer, a lubricant, a binder, a flavorant, and a coating.

22. The medical formulation of claim 20, wherein the formulation is delivered to an individual orally, nasally, inhalationally, parentally, intravenously, intraperitoneally, subcutaneously, intramuscularly, intradermally, topically, rectally, intracerebrally, intraventricularly, intracerebroventricularly, intrathecally, intracisternally, intraspinally, perispinally, intraocularly, or intravitreally.

23. The medical formulation of claim 20, wherein the 5′-UTR further comprises a spacer located between the translational enhancer and the coding region.

24. The construct of claim 23, wherein the spacer is approximately 100-150 nt in length.

25. The construct of claim 23, wherein the spacer is selected from the group consisting of: a Hoxa9 native spacer (SEQ ID NO: 15), an actin 5′-UTR (SEQ ID NO: 16), and an inverted actin 5′-UTR (SEQ ID NO: 17).

26. The construct of claim 20, wherein the translational enhancer is one or more of the following: a Hoxa9 IRES-like element, a stem-loop structure isolated from a Hox gene, SEQ ID NO: 1 or a sequence variant thereof, and SEQ ID NOs: 2-14.

27.-29. (canceled)

30. The construct of claim 20, wherein the translational enhancer possesses at least 75% sequence identity to SEQ ID NO: 1.


SEQUENCE LISTING (17 sequences)

SEQ ID NO: 1 - RNA, 35 nt, Mus musculus
cgcguuauuguucugccgggcggacacgugacgcg

SEQ ID NO: 2 - RNA, 35 nt, Artificial Sequence
  Mutated P4
cgcguuauucagcugccgggcggacacgugacgcg

SEQ ID NO: 3 - RNA, 35 nt, artificial sequence
  Mutated P4
cgcguuauuguucugccgggcguugacgugacgcg

SEQ ID NO: 4 - RNA, 35 nt, artificial sequence
  Mutated P4
cgcguuauucagcugccgggcguugacgugacgcg

SEQ ID NO: 5 - RNA, 35 nt, artificial sequence
  Mutated P4
cgcgaguguguucugccgggcggacacgugacgcg

SEQ ID NO: 6 - RNA, 35 nt, artificial sequence
  Mutated P4
cgcguuauuguucugccgggcggacacuauucgcg

SEQ ID NO: 7 - RNA, 35 nt, artificial sequence
  Mutated P4
cgcgaguguguucugccgggcggacacuauucgcg

SEQ ID NO: 8 - RNA, 35 nt, artificial sequence
  Mutated P4
cggcagauuguucugccgggcggacacgugacgcg

SEQ ID NO: 9 - RNA, 35 nt, Artificial Sequence
  Mutated P4
cgcguuauuguucugccgggcggacacguuugccg

SEQ ID NO: 10 - RNA, 35 nt, Artificial Sequence
  Mutated P4
cggcagauuguucugccgggcggacacguuugccg

SEQ ID NO: 11 - RNA, 35 nt, Artificial Sequence
  Mutated P4
cgcguuauuguucugggccgcggacacgugacgcg

SEQ ID NO: 12 - RNA, 35 nt, Artificial Sequence
  Mutated P4
cgcguuauuguucugaauugcggacacgugacgcg

SEQ ID NO: 13 - RNA, 18 nt, Artificial Sequence
  Mutated P4
gggcggacacgugacgcg

SEQ ID NO: 14 - RNA, 17 nt, Artificial Sequence
  Mutated P4
cgcguuauuguucugcc

SEQ ID NO: 15 - DNA, 144 bp, mus musculus
cgtggccaatgggggcgcgggcgccggcaacttattaggtgactgtacttcacccccccctggtgccaccaagttgttacatgaaatctgcagtttcataatttcggcgggtcgggctgggccggccaggcgcgggctactgca

SEQ ID NO: 16 - DNA, 109 bp, mus musculus
tataaaacccggcggcgcaacgcgcagccactgtcgagtcgcgtccacccgcgagcacagcttctttgcagctccttcgttgccggtccacacccgccaccagttcgcc

SEQ ID NO: 17 - DNA, 109 bp, mus musculus
ggcgaactggtggcgggtgtggaccggcaacgaaggagctgcaaagaagctgtgctcgcgggtggacgcgactcgacagtggctgcgcgttgcgccgccgggttttata